(19) United States
(12) Patent Application Publication
(10) Pub. No.: US 2010/0136171 A1
(43) Pub. Date: Jun. 3, 2010

(54) GOOD TASTING FOOD PRODUCT CONTAINING AN AGENT FOR REDUCING CARBOHYDRATE UPTAKE; AND COMPOSITIONS CONTAINING SUCH AN AGENT

(76) Inventors: Cornelis Margaretha Theodorus Maria Bongers, Helmond (NL); Renske Dekkers, Arnhem (NL); Petronella Johanna Looijesteijn, Zwolle (NL)

Correspondence Address: HOFFMANN & BARON, LLP, 6900 JERICHO TURNPIKE, SYOSSET, NY, 11791, US

(21) Appl. No.: 12/598,355

(22) PCT Filed: Apr. 9, 2008
(86) PCT No.: PCT/NL08/50198

§ 371 Date: Dec. 31, 2009

(30) Foreign Application Priority Data
May 1, 2007 (EP) 07107282.1

(51) Int. Cl. A23L 1/28; C12Q 1/02; C12N 1/20

(52) U.S. Cl. 426/61; 435/29; 435/252.9; 435/252.1

(57) ABSTRACT

The present invention relates to good tasting food products that contain an agent or agents which avoid that taste-essential ingredients having an adverse effect, when digested or when taken up in the system of the consumer, can perform their adverse effects. The said adverse effect is especially an adverse effect on human health. In addition, the present invention relates to particular compounds or compositions that can be used for the directed removal of such adverse components of foods (including drinks).

The present invention relates to good tasting food products that contain an agent or agents which avoid that taste-essential ingredients, and especially carbohydrates, having an adverse effect when digested or when taken up in the system of the consumer, can perform their adverse effect. The said adverse effect on human health is especially an adverse effect associated with too high calorie intakes. In addition, the present invention relates to particular compounds or compositions that can be used for the directed removal, and especially conversion, of such adverse components of foods (including drinks) especially in the upper intestinal tract. In addition, the present invention is directed to a process for obtaining microorganisms suitable for application in such food products, and to the use of microorganisms in converting sugar unities. In yet a further aspect, the present invention relates to some bacteria strains.

A well-known example of a taste-essential carbohydrate that also has adverse effects when taken up in the system of a consumer is sucrose. Sucrose is thé standard for a sweet taste, but has a high caloric value and, associated therewith, may lead to undesirable weight gains, obesity, diabetes type II etc.

Another example of such a carbohydrate is lactose which also imparts a pleasant sweetness, yet many persons suffer from lactose intolerance or allergy.

Quite a number of the adverse effects of carbohydrates in food products are associated with medical indications, such as obesitas, diabetes, allergic reactions and intolerances.

In the state of the art, a high number of products are known and described wherein the components having adverse effects are replaced by other harmless components. Sucrose may for example be replaced by artificial sweeteners. Oftentimes, however, replacement of the taste essential ingredient leads to a less pleasant or at least different or unfamiliar taste of the food product.

Also when part of the taste essential component is replaced by a taste intensifier, oftentimes less tasteful products result, the taste profile of a product changes or off-tastes appear.

The present invention has the object to provide a food, inclusive a drink, that maintains its pleasant taste, in the sense that the taste essential component, and especially mono- and disaccharides, maintains its sensoric availability, while the adverse effects thereof are reduced, alleviated, neutralized or sometimes even converted in favorable effects. That is, after providing their favorable taste effect, the components involved are converted in harmless and sometimes even health positive components.

Another is to provide compositions which can be consumed before or, preferably, after consumption of a food composition that contains an amount of taste essential components, and which compositions reduce, alleviate or neutralize adverse effects of said food composition or even convert said taste essential components in compounds having favorable effects.

### STATE OF THE ART

EP-0 457 919 relates to functional foods or edible compositions having a calorie intake lowering function. These compositions comprise at least one polysaccharide producing enzyme from the group consisting of glucosyl transferases and fructosyl transferases having water soluble polysaccharide production ability. The enzymes form polysaccharides from sucrose in the digestive tract to lower calorie intake and by this mechanism in the digestive tract, the food compositions described are said to be useful for the prophylaxis of adult disease factors such as obesity. To avoid reduction in activity by a digestive fluid such as the gastric juices when the enzyme is orally ingested, the compositions described are constituted in enteric dosage forms which protect the compositions against deactivation before the small intestine is reached.

In EP-B-0 968 719, an enzyme composition is described containing amylase and/or invertase together with an enzyme which is capable of forming an oligosaccharide in vivo, which oligosaccharide must exert physiological activity in the living body. When this enzyme composition is contacted with the food composition before consumption, the enzyme catalyzed reaction will start; dependent on the food composition this reaction may be more or less fast, but will have a negative effect on the taste of the food composition.

EP-A-0 075 604 teaches a method of lowering the blood glucose level in mammals and a blood glucose level-lowering agent. The method comprises administering an enzyme capable of synthesizing sparingly-digestible saccharides from easily-digestible saccharides. In the working examples sucrose is fed to mice that also consumed enzyme preparations. The pH values referred to in this application are around neutral, pointing to activity in the intestines.

Similar technology is described in WO-03/051391 teaching enzyme compositions provided with an enteric coating, which enzymes work in a pH range of 6-7.5.

WO-A-2004/080200 is directed to probiotic food compositions. These compositions contain spores of bacteria or fungi or contain enzymes, which active components should enhance the digestion of carbohydrates and/or proteins. To increase the probability of survival in gastro-intestinal conditions (low pH; bile) these microorganisms may be encapsulated or micro-encapsulated.

Finally, WO-2007/059955 teaches an agent for reducing the useable calorie content of food, and especially teaches to dehydrogenate fructose to 5-keto-D-fructose. In the working examples, the enzymes used are coated with a coating which is resistant to low pH values.

A problem with compositions having an enteric coating to protect the enzymes that need to convert the carbohydrate in compounds that are not taken up in the blood stream is—especially when used to inhibit the adverse effects of quickly absorbed mono- and disaccharides—that these have a very restricted area of action, being the upper part of the small intestines, where the carbohydrate containing food compositions only have a short residence time, in the sense that the carbohydrates to be converted will quickly be absorbed and be taken up in the body.

### INVENTION

The present invention solves the above-identified first object by combining taste essential mono- and/or disaccharides with a microorganism and/or an enzyme, which microorganism and/or enzyme is capable of converting in the stomach the mono- and/or disaccharide in a component or components which do not have the adverse effect or only have said effect in a lesser degree, said taste essential mono- and/or disaccharides and said microorganism and/or enzyme being present in said food without substantially reacting with each other before being consumed.

The present invention makes use of the storage capacity of the stomach. By starting to act in the stomach, a considerably higher amount of mono- and/or disaccharides are converted into compounds not having an adverse effect on the system of the consumer.

In a first embodiment, the invention hence relates to a food comprising (i) at least one taste essential mono- and/or disaccharide that has an adverse effect when taken up in the system of a consumer and (ii) at least one microorganism and/or enzyme capable of converting in the stomach ingredient (i) in a component or components not having said adverse effect or at least having said adverse effect in a decreased degree; wherein mono- and/or disaccharide (i) and microorganism and/or enzyme (ii) do not react with each other before consumption

In a preferred embodiment, the microorganism produces enzymes present in said food composition. Preferentially, said microorganism is a living microorganism, and more preferably a probiotic microorganism. Suitably, such a microorganism is selected from the genera Lactobacillus, Bifidobacterium, and Leuconostoc.

In yet a further preference, the enzyme is an exocellular enzyme. As an alternative, the enzyme is obtained from or in the form of a lysate of a microorganism.

In a second aspect, the present invention relates to a process for obtaining a microorganism suitable for application in the food of the invention, comprising selection of food grade microorganisms based on their glucose and fructose polymer formation from sucrose and mannitol formation from fructose under stomach conditions; incubating the selected microorganisms under stomach and intestine conditions in the presence of mono- and/or disaccharides; and subsequently selecting the microorganisms that are able to convert the mono- and disaccharides in a component or components not having an adverse effect or at least having said adverse effect in a decreased degree. Hence, those microorganism that survive the incubation conditions while producing said component or components not having said adverse effect or having it to a decreased degree, are harvested.

Food grade microorganisms are microorganisms which are safe to be used in food.

Polymer formation is determined by culturing microorganisms and especially bacteria in MRS-broth (broth according to De Man, Rogosa and Sharpe) containing sucrose at suitable conditions (for example, at 25° C. for Leuconostoc and at 37° C. for Lactobacillus strains). After growth, a small quantity of the cultured broth is put on MRS-agar plates containing 10% sucrose and incubated at a suitable growing temperature. Polymer producing bacteria appear as slimy colonies on these agar-plates. Besides the plate method, strains can also be screened for the presence of dextransucrase and levansucrase within their DNA with a dotblot method.

Screening for the ability to produce mannitol from fructose is done by using the enzyme mannitol-dehydrogenase. The following reaction occurs: D-fructose+NADH →D-mannitol+NAD. This method is monitored on the basis of the NADH to NAD conversion, which can suitably be measured at a wavelength of 340 nm using a spectrophotometer.

Further details are provided in Example 1 (vide infra).

In case in the remainder of this description, reference is made to “microorganism” as being a component of the food composition of the present invention, then the intention is to refer to said microorganism also as source of enzyme for use in said food composition according to the invention.

The microorganism and/or its enzymes to be used in the present invention have the possibility to work under conditions present in the stomach. That is, they work at a temperature around 37° C. and at acidic (to as low as a pH of 2-4) as well as neutral pH. By the ingestion of food, and especially protein rich food, the pH in the stomach will raise quickly to values between 5 and 7 and subsequently decreases to around 2 again. The microorganisms and/or their enzymes should essentially be active over this whole pH range in the stomach.

Increase in stomach pH could also be assured by incorporation of a buffering component in the formulation. In this way one assures an increase of the stomach pH irrespective of the composition of the food formulation. The buffering component could be e.g. salts like calcium phosphate, or proteins like casein or whey proteins.

The reaction between ingredient (i) and microorganism and/or enzyme (ii) only starts when the food according to the first aspect of the present invention, inclusive any drinks, is consumed. That is, the reaction takes place in the time span between the moment that the food or drink is in the mouth and the moment right before the taste essential component can create it adverse effect, for example the moment wherein the said ingredient is absorbed in the intestines. In accordance with the present invention, the reaction is at least carried out (or to the largest extent carried out) in the stomach.

By way of example, sucrose manifests its sweetness creating effect in the mouth, yet is converted in harmless or less harmful components before it is taken up by the small intestines in the system of the consumer where it may manifest its adverse effects as high calorie molecule and/or in diabetes type II.

With “taste essential mono- and/or disaccharide” is meant that an ingredient acceptable for use in a food, including a drink, has some characteristic effect on the taste of a product containing said ingredient. This characteristic may be a taste, an effect on the mouthfeel or any other perceivable sensoric effect. That is, a taste essential ingredient adds to the overall sensoric perception of a food.

In a preferred embodiment, said taste essential ingredient is selected from the group of carbohydrates and especially sucrose, fructose, lactose, glucose, galactose and maltose.

While carrying out the process of the present invention, the inventors have identified a number of bacteria strains, of which a few were deposited: Leuconostoc mesenteroides FF4028 (deposited as CBS 122667), Leuconostoc mesenteroides FF4029 (deposited as CBS 122668), Leuconostoc mesenteroides FF4152 (deposited as CBS 122669), and Lactobacillus reuteri FF6809 (deposited as CBS 122781). Furthermore, it was found that Leuconostoc mesenteroides ATCC 8393 is also capable of giving oligomers, but only after incubation of this microorganism at a pH of 3.0; incubation of this strain at pH 6.5 did not result in oligomer formation.

In yet a further aspect, the present invention relates to the use of a microorganism, which, when subjected to stomach conditions is induced to convert sugars in a component or components not having adverse effects or at least having adverse effects in a decreased amount (adverse effects in the sense of or as declared herein-above), for example by oligomerization or polymerization of sugar unities. Such a microorganism may be obtained by the above-described process of the invention.

Since not only the microorganisms per se, but also their enzymes may be used, the use according to the invention may also involve the recovery of its oligomerizing or polymerizing enzymes.

In a highly preferred embodiment, the microorganism or its enzymes are used in a digestive. Such a digestive may be used after consuming a meal containing a high amount of taste essential mono- and/or disaccharides. Of course, the time of consuming the digestive must be sufficiently close to the moment of consuming the meal, so that the major part of said meal is still present in the stomach. An advantage of such a digestive is that the effects of the present invention may be obtained without needing to adapt the food composition containing taste essential mono- and/or disaccharides. Suitably, the digestive is based on a dairy product, for instance a fermented dairy product, that does not contain too many mono- and/or disaccharides.

As a non-limiting example of a food ingredient that is essential for the taste of the food product, yet has adverse effects when taken up in the system of the consumer, sugars, and especially sucrose, will be discussed.

Carbohydrates are an important, if not the most important, source of energy in a food product and are found in many kinds in every day's food compositions. Within the carbohydrate family, sugars, and especially mono- and disaccharides, provide a pleasant sweet taste. To reduce the energetic or caloric value of sugars-containing, and especially of sucrose-containing compositions often artificial sweeteners are used. Such sweeteners however have oftentimes an adverse effect on the taste of the food product incorporating these. Moreover, large amounts of polyols (like xylitol) may give rise to flatulence or other systemic inconveniences. Furthermore, not all sweeteners are accepted by consumers nor by governmental authorities in all countries. In addition, the perception in taste may differ from consumer to consumer, while many consumers indicate that artificial sweetener containing compositions suffer from an unpleasant, for instance bitter or metallic, after-taste.

Food compositions having a high carbohydrate content may be categorized on the basis of the so-called glycemic index. This numeric index is based on the average increase of the glucose level in the blood of the consumer after eating a particular amount of the food composition. If a food composition contains a high amount of carbohydrates, then this does not necessarily mean that said composition automatically has a high glycemic index, since eating said composition may not lead to a higher glucose level. Instead of referring to the glycemic index, one also refers to the effective energetic value of a particular food composition. This value relates to the part of the energetic value of the food composition that is effectively absorbed by the system and gives energy in the form of calories.

The effective energetic value of a sugar containing food composition may be decreased when steps are taken to avoid or inhibit that the sugars (or their metabolic degradation products) are absorbed in the gastro-intestinal tract.

One way of achieving this is by inhibiting the hydrolytic activity of the enzyme sucrase in the intestines. Sucrase is an enzyme converting the disaccharide sucrose, also known as saccharose, in the monosaccharides fructose and glucose. An example of a sucrase inhibitor is L-arabinose.

Another known possibility, a possibility used in accordance with the present invention, is to convert digestible sugars in non-digestible molecules like oligosaccharides or sugar alcohols. Sugar alcohols are partially absorbed in the ileum, yet do not take part in the systemic metabolism and are excreted via the kidneys. The remaining part is slowly fermented in the colon, where these compounds may have a prebiotic activity, meaning that the growth and activity of certain beneficial bacteria in the intestinal flora, such as Lactobacilli and Bifidobacteria, are stimulated. Oligosaccharides have a corresponding behaviour in the intestines.

Particularly, commonly used digestible sugar molecules which may be converted by the microorganism and/or enzyme comprise, for example, sucrose, maltose, fructose, lactose, glucose and galactose; the said sugar molecules are converted to indigestible molecules like glucose polymer, fructose polymer, fructo-oligosaccharides (FOS), mannitol, and galacto-oligosaccharides (GOS). When, for instance, FOS or GOS is formed, it is known that such compounds have a prebiotic activity; that is, these compounds stimulate the growth and activity of benign bacteria, and especially of Lactobacilli and Bifidobacteria, in the intestinal flora. Hence, in a preferred embodiment of the present invention, at least part of the sugar molecules present in the food products of the invention are converted in oligosaccharides having prebiotic activity.

In accordance with the first aspect of the present invention, the reaction between the taste essential ingredient and the specific microorganism and/or enzyme is delayed until after the taste essential ingredient has displayed its taste effect; that is, the taste essential ingredient will be sensorically available, and only afterwards be converted or neutralized essentially starting in the stomach. Further, the amounts of the taste essential ingredient and the microorganisms and/or enzymes are mutually adjusted so that the taste essential ingredient is converted or neutralized to a predetermined, or at least acceptable or desired level under systemic conditions.

As an indication, the amount of enzymes to be used in a food composition of the present invention generally is at least about 0.1 wt. %, drawn on the mass of their substrate, for instance sugars; more preferably said amount is at least 0.2 or 0.5 wt. % and even more preferably at least about 1 wt. % drawn on the mass of the substrate. Similar amounts are needed in the use according to the invention. As an indication, for microorganisms to be used in a food composition of the present invention generally at least 10⁷ such as between 10⁷-10¹⁰ cells/ml are needed, more preferably between 10⁸-10⁹ cells/ml and even more preferably about 10⁸ cells/ml.

The microorganisms that may be used in the composition of the present invention can be living microorganisms, and are preferably microorganisms excreting exo-enzymes or containing cell-wall anchored enzymes, which enzymes are capable of converting the taste essential ingredient. Non-limitative examples are Leuconostoc and Lactobacillus sp. containing glucan and fructan sucrases that convert sucrose. In a more preferred embodiment, the microorganisms do not only provide desirable enzymes, but are themselves probiotics.

More in general, it is noted that examples of suitable enzymes to be used in the food compositions of the present invention are glucosyl transferases (for example dextran sucrase), fructosyl transferases (e.g., inulosucrase, levansucrase, fructan:fructan 6G-fructosyltransferase), galactosyl transferase, galactosidases and mannitoldehydrogenase.

Glucosyltransferases are enzymes involved in the production of various glucose polymers like dextran, mutan, alternan and reuteran. Glucosyltransferases may be produced by various lactic acid bacteria such as Lactobacillus reuteri, Lb. sakei, Lb. fermentum, Lb. parabuchneri, Streptococcus mutans, Streptococcus salivarius and Leuconostoc mesenteroides.

Fructosyltransferases may be produced by the following probiotics: Lactobacillus reuteri, Lactobacillus acidophilus, and Streptococcus salivarius. Also non-probiotic lactic acid bacteria such as Leuconostoc citreum, Leuconostoc mesenteroides and Lactobacillus sanfranciscensis and microorganisms like Gluconacetobacter sp., Zymomonas mobilis, Arthrobacter sp., Aspergillus niger and Bacillus sp. are able to produce fructosyltransferases. A highly preferred enzyme is the levansucrase from Lactobacillus reuteri 121 because it shows a relatively high degree of sucrose conversion. In addition, it is known that fructosyltransferases occur in high concentrations in among others, onions and chicory. The synthesis of fructosyltransferases by bacteria may be increased by preculturing the microorganisms in a medium containing sucrose.

Mannitoldehydrogenases show a very efficient conversion of fructose to mannitol. These enzymes may be produced by the following probiotics: Lactobacillus intermedius, Lactobacillus fermentum, Lactobacillus reuteri, and Lactobacillus plantarum. Also non-probiotic bacteria like Leuconostoc pseudomesenteroides, Leuconostoc mesenteroides and Lactobacillus sanfranciscensis are capable of producing mannitoldehydrogenases. The activity of mannitoldehydrogenases may be increased by preculturing the microorganisms in a medium containing fructose.

Galactosidases may be produced by the probiotic Bifidobacterium adolescentis (α-galactosidase). Also non-probiotic microorganisms like Kluyveromyces fragilis, Aspergillus oryzae and Escherichia coli can produce galactosidases (β-galactosidase). In this light, it is noted that β-galactosidases are preferred because of the production of oligomers with prebiotic activity. Instead of living microorganisms also dead organisms may be used as a kind of reservoir for suitable enzymes. In order to make these enzymes available, it may sometimes be needed to use disrupted or lysated microorganisms.

In the most preferred embodiment, the microorganisms are lactic acid bacteria, such as those of the genera Leuconostoc or Lactobacillus, especially because these microorganisms are often used in food applications.

Instead of or in combination with microorganisms also enzymes may be used. Such enzymes may be of botanic origin (e.g. of chicory), of microbial origin (e.g. levansucrase from Zymomonas mobilis or transglucosidase from Aspergillus niger) or of animal origin.

In one embodiment, the microorganisms and/or enzymes are physically separated from the taste essential ingredient in the food composition, or the two reactants are because of physical and/or chemical conditions not capable to react with each other in the unconsumed food composition.

This may be achieved by incorporating or encapsulating the microorganisms and/or enzymes in a solid (inclusive of gel-type) matrix such that interaction with the taste essential ingredient becomes only possible after consumption of the food product. For instance can the matrix be disrupted by chewing, by the action of saliva or of other gastrointestinal fluids, by peristaltic movements, by the kneading action of the stomach and so on. Suitable carriers encompass liposomes and specific adsorbants, such as cyclodextrins or molecularly imprinted polymers. The encapsulation should allow the microorganism or its enzymes to start acting on the aimed substrate already in the stomach.

Instead of using a solid matrix, the taste essential ingredient and the microorganism and/or enzyme may also be present in different phases of an emulsion or even a double emulsion.

Also combinations of both types, such as encapsulated double emulsions made according to the teaching of e.g. EP-A-1 324667, may be used, which system guarantees the survival of probiotic bacteria.

Dependent on the type of enzyme and/or probiotic, the optimal activity is achieved at a temperature in the range of 25-60° C. Preferably the enzymes and/or bacteria are selected so that the optimal activity is achieved at a temperature between 35 and 40° C.; that is, close to body temperature. By encapsulating the enzymes and/or microorganisms, the latter are not in contact with the substrate being present in the food composition, which makes it possible, of course also dependent on the other constituents of the food composition, to store the product at ambient temperature.

Another mechanism for inhibiting the reaction between the microorganism and/or enzyme and the taste essential ingredient is the presence of an inhibitor, and especially a high molecular weight inhibitor, for said reaction. After consumption of the food product encompassing both reactants, the constituents of the food composition are diluted, allowing the interaction between the reactants.

Microorganisms and/or enzymes may be used requiring a specific pH or a specific compound associated with or present in the stomach, especially the low pH as it exists in the stomach, generally a pH in the range of 2-4. Particularly, the invention also relates to the use of a microorganism, which when subjected to stomach conditions is induced to oligomerise or polymerise sugar unities, in the oligomerization or polymerization of sugar unities.

An example of such a microorganism is Leuconostoc mesenteroides strain FF4003 (ATCC 8393) as used in the working examples (vide infra).

In yet a further embodiment the temperature of the food composition is below the temperature needed for the reaction to occur.

The food composition per se may also have a pH that is too high or too low for allowing the reaction, which condition changes upon consuming said food composition, for example by dilution. Alternatively, the water activity and/or the salt strength of the surrounding food composition may avoid or inhibit the reaction to occur.

Of course, also combinations of the possibilities discussed in the previous paragraphs may be made, such as using a matrix based on calcium phosphate, which salt may lead to an increased pH in the stomach, which increased pH may be associated with an ideal environment for specific microorganisms and/or enzymes.

The most preferred embodiments lead to the conversion of said ingredients having an adverse effect into components that have a beneficial effect in the human or animal body, such as health improving effects.

Generally, it has advantages to use microorganisms instead of enzymes in the food compositions of the present invention. Microorganisms are less expensive than enzymes, and may provide a suitable environment for the reaction of the enzymes prepared by them.

In case probiotics are additionally present in the food compositions of the invention, it is of course of importance that at least part of these probiotics are activated in the guts. That is, it is required that the said bacteria survive the conditions in the stomach. If the probiotics do not have the property of being resistant to the stomach pH, then these may be coated.

One can use bacteria that are cultured in an acidic medium to achieve an improved resistance to an acidic pH. By such culture step, the activity of enzymes produced by such bacteria at lower pH values may increase, as well.

Where the first embodiment of the present invention has the taste essential ingredient and the microorganism and/or enzyme present in a food composition, the present invention also relates to a food composition comprising the microorganism and/or enzyme to be used before or after consumption of a food product containing a (potentially present) taste essential ingredient. That is, the food composition comprising the microorganism and/or the enzyme is used profylactically or therapeutically, with the aim of countering the adverse effects of said taste essential ingredients.

If a consumer consumed a particular ingredient which may have adverse effects, or expects to consume such an ingredient, he may consume a food composition comprising a microorganism and/or enzyme to counter the negative effects of such ingredients.

An example of this embodiment of the invention is a food composition, for instance a fermented milk product, such as a yoghurt, buttermilk or cheese (product), comprising transglycosylation enzymes and/or mannitol dehydrogenase derived from Lactobacillus sp., Leuconostoc sp., Streptococcus sp. or Bifidobacteria, which enzymes may become active in the gastric environment.

Especially for the embodiment wherein the food composition comprises the microorganism and/or enzyme to be used before consumption of a food product containing a (potentially present) taste essential ingredient, it may be advantageous to delay the passage through the gastro-intestinal tract, and especially through the upper intestinal tract. Such a delay may be achieved by having the microorganisms and/or enzymes present in a controlled release embodiment, such as a film, or in an adhering matrix, such as gel particles that stick to the wall of the oral cavity, or pharynx or oesophagus.

Suitable foods, including beverages, incorporating the present invention may be fruit juices, soft drinks, dairy product, candy and chocolate bars, cakes etc. Such products are preferably packed to provide a food composition having an acceptable storage stability and keepability. The steps discussed above to avoid reaction between the enzymes and/or microorganisms and their substrates need to be taken in view of such packed products.

In yet a further aspect, the present invention relates to a process to prepare the food composition of the invention. Such a product may be prepared by adding at any time during the preparation of the food composition, yet preferably after a pasteurization or sterilization step, a supplement containing the enzymes and/or microorganisms capable of converting the taste essential ingredient.

The invention will now be elaborated while referring to the following, non-limiting examples.

### EXAMPLE 1

### Screening Method for Suitable Strains

Screening of suitable micro organisms for the conversion of sucrose and fructose to oligomers and mannitol is done according to the following procedure:

1. Screening of polymer production by microorganisms from sucrose by:

a plate method: bacteria (Leuconostocs and lactobacilli) are grown on agar plates containing sucrose. Bacteria that are able to produce polymers from sucrose appear as slimy colonies on these plates. The slimy colony producing strains are selected;

a dot blot method: this method looks at the presence of specific genes coding for enzymes such as dextransucrase, levansucrase, inulosucrase. Microorganisms that contain these genes are selected.

2. Screening of mannitol production from fructose by an enzyme assay: the oxidation of NADH to NAD⁺ that coincides during the conversion of fructose to mannitol is spectrophotometrically measured at pH 5.3 at a wavelength of 340 nm. Strains from which the slope at 340 nm divided by the optical density (OD) at 600 nm is smaller than −0.2 are selected. The OD at 600 nm is preferably between 0.150 and 0.350 and is a measure for the amount of cells in the assay.

3. Suitable strains from former two steps are incubated at successively pH 3 and pH 6.5 with sucrose and fructose for a certain time to mimic digestion. Oligomer and mannitol production are monitored by LC-analysis. Before incubation, strains are pre-cultured in a medium where sucrose and fructose are present. Strains that produce oligosaccharides and/or mannitol after the pH 3 incubation step are selected.

Based on the outcome of step 1, 2, and 3, strains that produce a suitable amount of oligomers and/or mannitol during the incubation are selected for further testing.

### EXAMPLE 2

Three selected strains, Leuconostoc mesenteroides FF4028 (deposited as CBS 122667), FF4029 (deposited as CBS 122668) and FF4152 (deposited as CBS 122669) are incubated for 1 hour at pH 3 followed by 1 hour at pH 6.5, the oligomer production was determined with LC.

Two other strains are incubated for 15 minutes at pH 3.0, for 1 hour at pH 6.5 and 15 minutes at pH 3.0 followed by 1 hour pH6.0. Strain FF4003 (ATCC 8393) is a Leuconostoc mesenteroides which oligomer production is induced by the incubation at pH 3.0. Incubation at pH 6.5 alone does not give oligomer; however incubation at pH 3.0 followed by incubation at pH 6.5 does give oligomers. This strain needs the stomach pH to produce oligomers. Strain FF6809 (deposited as CBS 122781) is a Lactobacillus reuteri that is not influenced by the different pH's, all three different incubations give the same chromatogram.

For the qualitative analysis of formed oligosaccharides and polysaccharides, a Dionex System is used equipped with Chromeleon software (Dionex, Sunnyvalley USA).

This system consists of a:
Dionex quaternary GP40 eluent pump to create a gradient of increasing NaOH and Na-Acetate concentration;
Dionex CarboPac™ PA-1 guard column (4*50 mm) P/N 43096;
Dionex CarboPac™ PA-1 analytical column (4*250 mm) P/N 35391;
Dionex ED40 electrochemical detector;
Dionex AS40 autosampler; and
Dionex software Chromeleon release 6.4.

Chemicals for preparing eluents:
Degassed milli Q water;
NaOH solution 50%; and
Sodium Acetate (anhydrous).

Eluents:
A; 12.5 mMol NaOH/l;
B; 8 mMol Na-Acetate/l;
C; 125 mMol NaOH/1; and
D; 500 mMol Na-Acetate in 125 mMol NaOH/l.
Eluent flow 1 ml/min
Column temperature 25° C.

The following gradient is used

| Time in | | | | | | mM Na- |
|---|---|---|---|---|---|---|
| minutes | % A | % B | % C | % D | mM NaOH | Acetate |
| 0 | 70 | 30 | | | 9 | 2 |
| 5 | 70 | 30 | | | 9 | 2 |
| 20 | 80 | 30 | 15 | 5 | 35 | 25 |
| 45 | 20 | | 45 | 30 | 97 | 150 |
| 50 | | | | 100 | 125 | 500 |
| 60 | | | | 100 | 125 | 500 |
| 61 | 70 | 30 | | | 9 | 2 |
| 80 | 70 | 30 | | | 9 | 2 |

Samples of 25 μl are injected on the system by an eluent flow of 1 ml/min and the chromatogram is recorded. Mono- and disaccharides are shown in the chromatograms as peaks with a retention time up to 18 minutes. Oligo's and polymers are shown as peaks with a retention time from 18 up to 50 minutes. Time 50 to 80 minutes is used to equilibrate the column for the next analysis.

All samples were 10 times diluted prior to analysis. The chromatograms were corrected for the blank sugar solutions.

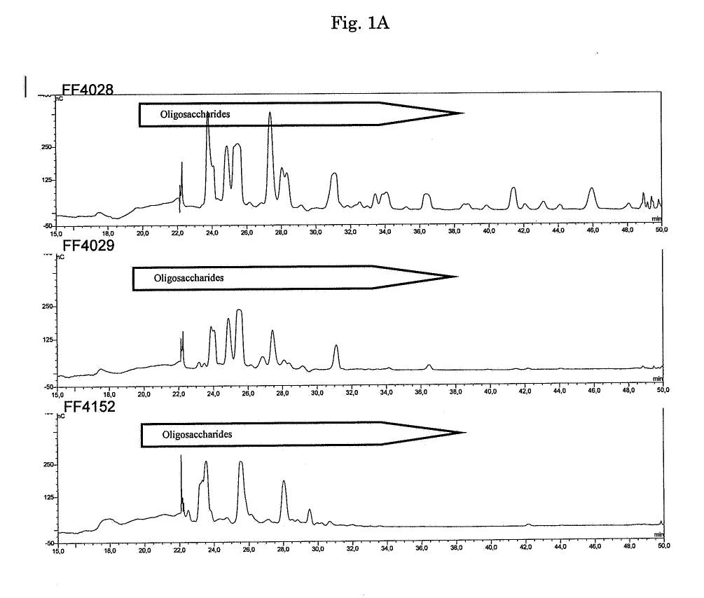

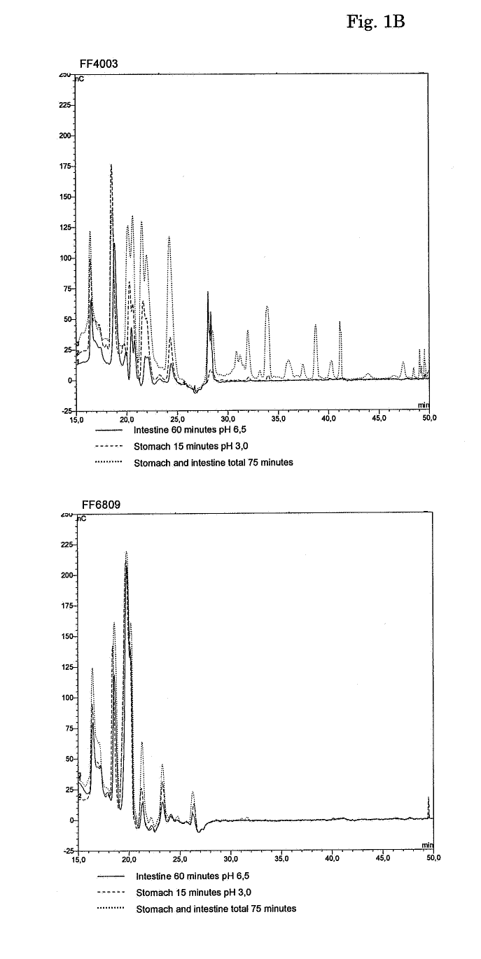

The strains showed a good oligomer production under the tested circumstances as can be seen from FIG. 1 showing the results of the LC. The sugar conversion to oligomers of the shown incubations was around 14-17%; when a combination of the three strains was used this conversion increased to 25-30%.

### EXAMPLE 3

The blood glucose concentration of 3 healthy volunteers was measured after ingestion of 50 gram of sugars. The measurements have been done four times: two times when the sugars were ingested and two times after the ingestion of the sugars; 50 ml of buffer with Lactobacillus reuteri FF6809 was also ingested.

The fourth volunteer was an individual with diabetes type 1. This person ingested 25 gram of sugars the first time and the second time after the ingestion of sugars also micro organisms were ingested.

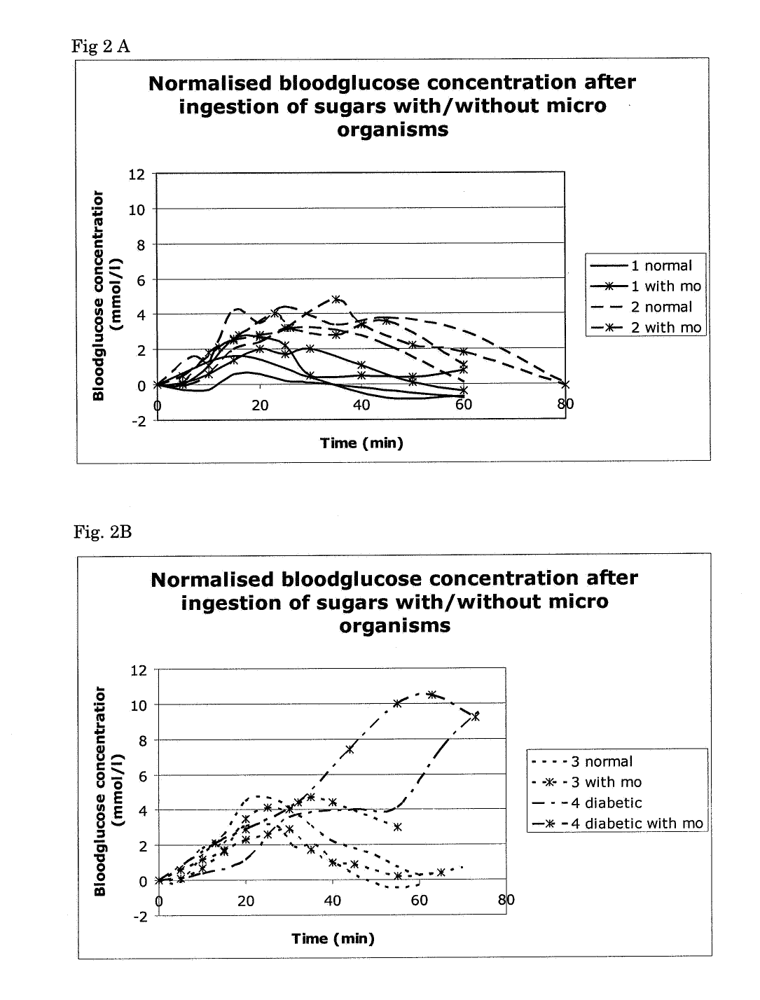

The blood glucose concentrations of all persons were normalised. It can be seen from FIG. 2 that in healthy volunteers the ingestion of microorganisms after a sugar dose does not affect the glucose concentration in the blood. The insulin response on the amount of sugars taken up in the blood is regulated very efficiently so a decrease in sugar uptake in case of the ingestion of micro organisms can not be determined by measuring the glucose content in the blood. However, in a person with diabetes type 1, where there is no insulin response, one can see that in case of the ingestion of only sugars more sugars are taken up in the blood when compared with the test in which micro organisms are ingested after the sugars. This is a strong indication that the micro organisms also do their work in vivo.

1. Food comprising (i) at least one taste essential mono- and/or disaccharide that has an adverse effect when taken up in the system of a consumer and (ii) at least one microorganism and/or enzyme capable of converting in the stomach ingredient (i) in a component or components not having said adverse effect or at least having said adverse effect in a decreased degree; wherein mono- and/or disaccharide (i) and microorganism and/or enzyme (ii) do not react with each other before consumption.

2. Food composition according to claim 1, wherein said taste essential mono- and/or disaccharide is a taste compound or has an effect on the mouthfeel.

3. Food composition according to claim 1, wherein said microorganism produces an enzyme that is selected from the group consisting of the glucosyl transferases, including dextran sucrase; fructosyl transferases, including inulosucrase, levansucrase, fructan:fructan 6G-fructosyltransferase; galactosyl transferase, and mannitoldehydrogenase.

4. Food composition according to claim 3, wherein the microorganism is a living microorganism, and preferably a probiotic microorganism.

5. Food composition according to claim 3, wherein the microorganism is selected from the genera Lactobacillus, Bifidobacterium, and Leuconostoc.

6. Food composition according to claim 3, wherein the enzyme is an exocellular enzyme.

7. Food composition according to claim 1, wherein the enzyme is obtained from or is in the form of a lysate of a microorganism.

8. Food composition according to claim 1, wherein mono- and/or disaccharide (i) and microorganism or enzyme (ii) do not react with each other before consumption, because the microorganism or enzyme is encapsulated in a matrix material to be disrupted during or after consumption.

9. Food composition according to claim 1, wherein mono- and/or disaccharide (i) and microorganism or enzyme (ii) do not react with each other before consumption, because of the physical and/or chemical conditions in the unconsumed composition are such that the microorganism or enzyme is not or hardly capable of reacting with ingredient (i).

10. Food composition according to claim 9, wherein the physical and/or chemical conditions are selected from the group consisting of a non-optimal temperature, a reversible enzyme inhibitor, a non-optimal pH, the absence of an activator for the reaction between (i) and (ii), said activator being present in the system of the consumer.

11. Food composition according to claim 1, being a dairy product, including a fermented milk product, such as a yoghurt, buttermilk, cheese.

12. Process for obtaining a microorganism suitable for application in the food of the invention, comprising selection of food grade microorganisms based on their glucose and fructose polymer formation from sucrose and mannitol formation from fructose under stomach conditions; incubating the selected microorganisms under stomach and intestine conditions in the presence of mono- and/or disaccharides; and subsequently selecting the microorganisms that are able to convert the mono- and disaccharides in a component or components not having an adverse effect or at least having said adverse effect in a decreased degree.

13. The process of claim 12, wherein the mono- and/or disaccharide is selected from sucrose, lactose, maltose, fructose, glucose and galactose.

14. The process of claim 12, wherein it is determined that oligomers and/or mannitol are formed.

15. Use of a microorganism, which, when subjected to stomach conditions is induced to convert sugars in a component or components not having adverse effects or at least having adverse effects in a decreased amount, for example by oligomerization or polymerization of sugar unities.

16. Use of a microorganism, which, when subjected to stomach conditions is induced to convert sugars in a component or components not having adverse effects or at least having adverse effects in a decreased amount, for example by oligomerization or polymerization of sugar unities, wherein the microorganism is obtainable by the process of claim 12.

17. Use according to claim 15, involving the recovery of oligomerizing or polymerizing enzymes.

18. Use according to claim 15, wherein the microorganism or its enzymes are used in a digestive.

19. Leuconostoc mesenteroides FF4028 (deposited as CBS 122667), Leuconostoc mesenteroides FF4029 (deposited as CBS 122668), Leuconostoc mesenteroides FF4152 (deposited as CBS 122669), and Lactobacillus reuteri FF6809 (deposited as CBS 122781).
